(19) United States
(12) Patent Application Publication
(10) Pub. No.: US 2023/0085093 A1
(43) Pub. Date: Mar. 16, 2023

(54) COMPUTERIZED PREDICTION OF HUMERAL PROSTHESIS FOR SHOULDER SURGERY

(71) Applicant: Howmedica Osteonics Corp., Mahwah, NJ (US)

(72) Inventors: Jean Chaoui, Locmaria Plouzané (FR); Gilles Walch, Lyon (FR); Manuel Jean-Marie Urvoy, Brest (FR)

(21) Appl. No.: 17/799,172

(22) PCT Filed: Feb. 18, 2021
(86) PCT No.: PCT/US2021/018533

§ 371 Date: Aug. 11, 2022

Related U.S. Application Data
(60) Provisional application No. 62/978,103, filed on Feb. 18, 2020.

(51) Int. Cl. A61B 34/10; A61F 2/46; G06N 20/00; G16H 30/20

(52) U.S. Cl. CPC A61B 34/10; A61F 2/4612; G06N 20/00; G16H 30/20; A61B 2034/108; A61B 2034/105; A61B 2034/102; A61F 2002/4633

(57) ABSTRACT

A surgical assistance system obtains patient-specific values of a plurality of physical characteristics of a humeral bone of a patient. The surgical assistance system predicts, based on the patient-specific values, a humeral prosthesis (202) for the patient from among a plurality of humeral prostheses, wherein the predicted humeral prosthesis has a filling ratio less than a remodeling threshold for the patient. The filling ratio of the humeral prosthesis is a ratio of (i) a radial distance from a lengthwise central axis of a stem (206) of the humeral prosthesis to an outer surface of the stem, to (ii) a radial distance from the lengthwise central axis of the stem to an inner surface of the intramedullary canal (208) of a humerus of the patient. The remodeling threshold for the patient is a filling ratio above which the humeral prosthesis would cause remodeling in the patient.

This application claims priority to U.S. Provisional Patent Application 62/978,103, filed Feb. 18, 2020, the entire content of which is incorporated by reference.

### BACKGROUND

Specific types of shoulder repair surgery involve inserting a stemmed humeral prosthesis into an intramedullary canal of a humerus of a patient. The intramedullary canal of the humerus is a space defined within the cortical bone along the humerus shaft. The stemmed humeral prosthesis may replace the humeral head of the humerus. In examples where a total anatomic shoulder arthroplasty or a hemiarthroplasty is performed, the stemmed humeral prosthesis may have a ball-shaped surface that slides within a concave-shaped surface of a glenoid implant or the patient's natural glenoid. In examples where a total reverse shoulder arthroplasty is performed, a ball-shaped glenoid implant slides within a concave-shaped surface of the stemmed humeral prosthesis.

Because different patients have differently sized and shaped bones, a surgeon needs to select an appropriately sized and shaped humeral implant for the patient. Selecting a humeral prosthesis that is too small may lead to loosening of the humeral prosthesis within the patient's humerus. Selecting a humeral prosthesis that is too large may lead to fracturing of the patient's humerus. Selecting a humeral prosthesis that is too large may also lead to bone remodeling and/or stress shielding.

### SUMMARY

This disclosure describes example techniques for predicting humeral prostheses to use with specific patients. As described herein, a surgical assistance system may obtain patient-specific values of a plurality of physical characteristics of a humeral bone of a patient. Additionally, the surgical assistance system may predict, based on the patient-specific values, a humeral prosthesis for the patient from among a plurality of humeral prostheses, wherein the predicted humeral prosthesis has a filling ratio less than a remodeling threshold for the patient.

In one example, this disclosure describes a method comprising: obtaining, by a surgical assistance system, patient-specific values of a plurality of physical characteristics of a humeral bone of a patient; and predicting, by the surgical assistance system, based on the patient-specific values, a humeral prosthesis for the patient from among a plurality of humeral prostheses, wherein the predicted humeral prosthesis has a filling ratio less than a remodeling threshold for the patient, wherein: the filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient, and the remodeling threshold for the patient is a filling ratio above which the predicted humeral prosthesis would cause bone remodeling in the patient. In another example, this disclosure describes a computing system comprising: a memory configured to store medical imaging data; and processing circuitry configured to perform this method. In another example, this disclosure describes a computing system comprising: means for storing medical imaging data; and means for performing this method. In another example, this disclosure describes a computer-readable data storage medium having instructions stored thereon that, when executed, cause a computing system to perform this method.

In another example, this disclosure describes a method comprising: obtaining, by a surgical assistance system, patient-specific values of a plurality of patient-specific values, wherein: the plurality of patient-specific values includes patient-specific values of a plurality of physical characteristics of a humeral bone of a patient, and the plurality of physical characteristics includes (i) an average cortical metaphyseal bone density in Hounsfield units that exceed a first threshold, (ii) an average cortical spongious bone density in Hounsfield units that are less than or equal to a second threshold, and (iii) one or more shape parameters of a statistical shape model (SSM) of the humeral bone of the patient; and predicting, by the surgical assistance system, based on the patient-specific values, a humeral prosthesis for the patient from among a plurality of humeral prostheses.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example surgical assistance system that may implement the techniques of this disclosure.

FIG. 2 is a conceptual diagram illustrating an example proximal portion of a humerus.

FIG. 3 is a flowchart illustrating an example operation of the surgical assistance system, in accordance with one or more aspects of this disclosure.

### DETAILED DESCRIPTION

As noted above, because different patients have differently sized and shaped bones, a surgeon needs to select an appropriately sized and shaped humeral implant for the patient. Selecting a humeral prosthesis that is too small may lead to loosening of the humeral prosthesis within the patient's humerus. Selecting a humeral prosthesis that is too large may lead to fracturing of the patient's humerus.

In addition to problems such as loosening of the humeral prosthesis or fracturing of the humerus, a humeral prosthesis may cause bone remodeling of the humerus if a filling ratio of the humeral prosthesis is too great. Bone remodeling may also occur due to other reasons, such as a misalignment of a stem axis of a humeral prosthesis and a humerus shaft axis. The filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient. Bone remodeling is a process in which the patient's body naturally resorbs bone tissue from specific locations and may grow bone tissue at other locations. Bone remodeling may lead to weakening of specific locations on the patient's humerus, which may eventually lead to a humeral fracture. Bone remodeling is especially a risk if the patient already has lowered bone density in particular areas of the patient's humerus. Accordingly, it is important to account for the potential of bone remodeling when selecting a humeral implant for a patient. A remodeling threshold for the patient is a filling ratio above which a humeral prosthesis would cause bone remodeling in the patient.

Because there may be many humeral prostheses from which to choose, it may be difficult for a surgeon to select an appropriate humeral prosthesis for a specific patient. Accordingly, it may be desirable to build computerized tools to help the surgeon select appropriate humeral prostheses for specific patients. However, it is unclear how such computerized tools may in fact be implemented.

In accordance with one or more techniques of this disclosure, a surgical assistance system may obtain patient-specific values of a plurality of physical characteristics of a humeral bone of a patient. The surgical assistance system may predict, based on the patient-specific values, a humeral prosthesis for the patient from among a plurality of humeral prostheses. The predicted humeral prosthesis has a filling ratio less than a remodeling threshold for the patient.

There are many possible types of physical characteristics that may be used as a basis for predicting a humeral prosthesis for a specific patient. However, use of too many physical characteristics or marginally relevant physical characteristics may impose significant data storage costs, processing costs, and/or bandwidth costs on a surgical assistance system. Furthermore, use of too many physical characteristics may decrease how quickly the surgical assistant system may determine the predicted surgical prosthesis. At the same time, use of too few physical characteristics or use of low-value physical characteristics may lead to inconsistent results.

In accordance with one or more techniques of this disclosure, a region at a diaphysis of the humeral bone is partitioned into a first set of blocks and a region at a metaphysis of the humeral bone is partitioned into a second set of blocks. As part of obtaining the patient-specific values for the plurality of physical characteristics, the surgical assistance system may calculate a first value as an average of Hounsfield units of the first set of blocks that exceed a first threshold, calculate a second value as an average of Hounsfield units of the second set of blocks that exceed a second threshold, and calculate a third value as an average of Hounsfield units of the first set of blocks that exceed a third threshold different from the first threshold. Use of these three values may enable surgical assistance systems to generate, in a computationally efficient manner, reliable predictions for humeral implants without reliance on a large number of physical characteristics.

In some examples, the surgical assistance system may obtain patient-specific values of a plurality of patient-specific values. The plurality of patient-specific values may include patient-specific values of a plurality of physical characteristics of a humeral bone of a patient. The plurality of physical characteristics may include (i) an average cortical metaphyseal bone density in Hounsfield units that exceed a first threshold, (ii) an average cortical spongious bone density in Hounsfield units that are less than or equal to a second threshold, and (iii) one or more shape parameters of a statistical shape model (SSM) of the humeral bone of the patient. The surgical assistance system may predict, based on the patient-specific values, a humeral prosthesis for the patient from among a plurality of humeral prostheses. Use of this combination of factors may provide a computationally efficient way of predicting the humeral prosthesis.

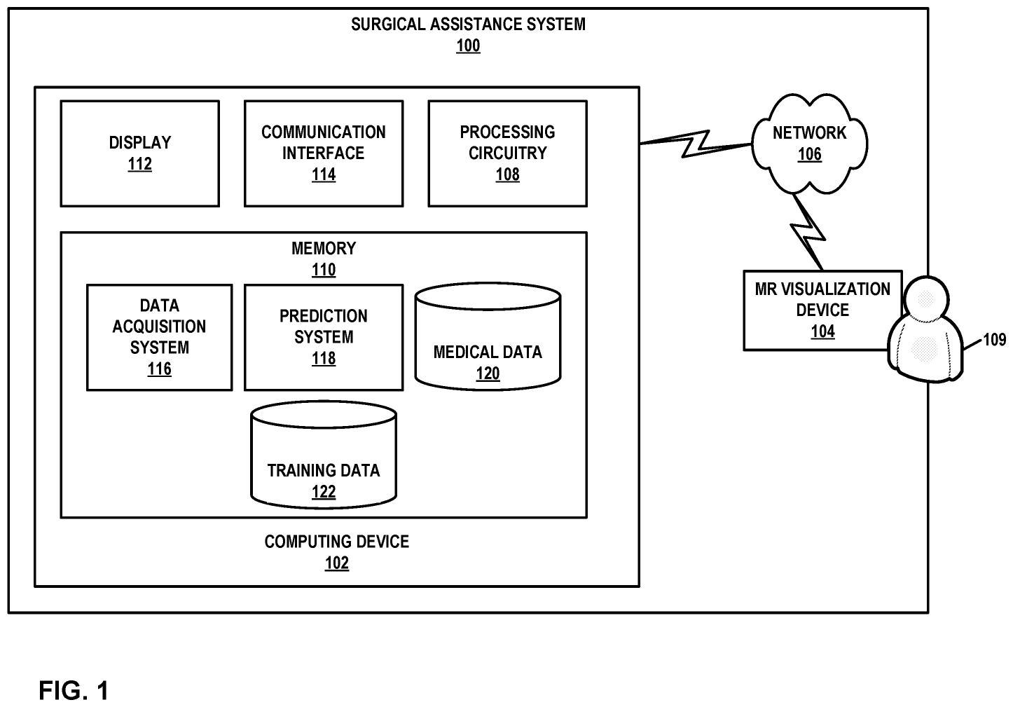

FIG. 1 is a block diagram illustrating an example surgical assistance system 100 that may be used to implement one or more techniques of this disclosure. Surgical assistance system 100 is an example of a computing system that may implement the techniques of this disclosure. Surgical assistance system 100 includes a computing device 102, a MR visualization device 104, and a network 106. In other examples, surgical assistance system 100 may include more or fewer devices or systems. For instance, in some examples, tasks performed by computing device 102 may be performed by MR visualization device 104. In other examples, tasks described in this disclosure as being performed by computing device 102 may be performed by a system of multiple computing devices. Furthermore, in some examples, surgical assistance system 100 may include multiple MR visualization devices or may include no MR visualization devices. In some examples, surgical assistance system 100 does not include network 106.

In the example of FIG. 1, memory 110 stores instructions for a data acquisition system 116 and a prediction system 118. Execution of the instructions for data acquisition system 116 by processing circuitry 108 may cause computing device 102 to provide the functionality ascribed in this disclosure to data acquisition system 116. Execution of the instructions for prediction system 118 by processing circuitry 108 may cause computing device 102 to provide the functionality ascribed in this disclosure to prediction system 118.

Computing device 102 of surgical assistance system 100 may include various types of computing devices, such as server computers, personal computers, smartphones, laptop computers, and other types of computing devices. In the example of FIG. 1, computing device 102 includes processing circuitry 108, memory 110, display 112, and a communication interface 114. Display 112 is optional, such as in examples where computing device 102 comprises a server computer.

Examples of processing circuitry 108 include one or more microprocessors, digital signal processors (DSPs), application specific integrated circuits (ASICs), field programmable gate arrays (FPGAs), hardware, or any combinations thereof. In general, processing circuitry 108 may be implemented as fixed-function circuits, programmable circuits, or a combination thereof. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Processing circuitry 108 may include arithmetic logic units (ALUs), elementary function units (EFUs), digital circuits, analog circuits, and/or programmable cores, formed from programmable circuits. In examples where the operations of processing circuitry 108 are performed using software executed by the programmable circuits, memory 110 may store the object code of the software that processing circuitry 108 receives and executes, or another memory within processing circuitry 108 (not shown) may store such instructions. Examples of the software include software designed for surgical planning. Processing circuitry 108 may perform the actions ascribed in this disclosure to surgical assistance system 100.

Memory 110 may store various types of data used by processing circuitry 108. For example, memory 110 may store medical data 120. Medical data 120 may include data describing 3D models of various anatomical structures, including morbid and predicted premorbid anatomical structures. For instance, in one specific example, medical data 120 may include data describing a 3D model of a humerus of a patient, imaging data, and other types of data. In the example of FIG. 1, memory 110 may also include training data 122.

Memory 110 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 112 may include a liquid crystal display (LCD), a plasma display, an organic light emitting diode (OLED) display, or another type of display device.

Communication interface 114 of computing device 102 allows computing device 102 to output data and instructions to and receive data and instructions from MR visualization device 104 via network 106. Communication interface 114 may be hardware circuitry that enables computing device 102 to communicate (e.g., wirelessly or using wires) with other computing systems and devices, such as MR visualization device 104. Network 106 may include various types of communication networks including one or more wide-area networks, such as the Internet, local area networks, and so on. In some examples, network 106 may include wired and/or wireless communication links.

MR visualization device 104 may use various visualization techniques to display MR visualizations to a user 109, such as a surgeon, nurse, technician, or other type of user. A MR visualization may comprise one or more virtual objects that are viewable by a user at the same time as real-world objects. Thus, what user 109 sees is a mixture of real and virtual objects. It is noted that user 109 does not form part of surgical assistance system 100.

MR visualization device 104 may comprise various types of devices for presenting MR visualizations. For instance, in some examples, MR visualization device 104 may be a Microsoft HOLOLENS™ headset, available from Microsoft Corporation, of Redmond, Wash., USA, or a similar device, such as, for example, a similar MR visualization device that includes waveguides. The HOLOLENS™ device can be used to present 3D virtual objects via holographic lenses, or waveguides, while permitting a user 109 to view actual objects in a real-world scene, i.e., in a real-world environment, through the holographic lenses. In some examples, MR visualization device 104 may be a holographic projector, head-mounted smartphone, special-purpose MR visualization device, or other type of device for presenting MR visualizations. In some examples, MR visualization device 104 includes a head-mounted unit and a backpack unit. In other examples, all functionality of MR visualization device 104 is performed in a head-mounted unit.

MR visualization device 104 may use visualization tools that are available to utilize patient image data to generate 3D models of bone contours to facilitate preoperative planning for joint repairs and replacements. These tools allow surgeons to design and/or select surgical guides and implant components that closely match the patient's anatomy. These tools can improve surgical outcomes by customizing a surgical plan for each patient. An example of such a visualization tool for joint repair surgeries is the BLUEPRINT™ system available from Wright Medical Technology, Inc. The BLUEPRINT™ system provides the surgeon with two-dimensional planar views of the joint-repair region as well as a three-dimensional virtual model of the joint-repair region. The surgeon can use the BLUEPRINT™ system to select, design or modify appropriate orthopedic prostheses, determine how best to position and orient the orthopedic prostheses, how to shape the surface of bones to receive the orthopedic prostheses, and how to design, select or modify surgical guide tool(s) or instruments to carry out a surgical plan. The information generated by the BLUEPRINT™ system may be compiled in a preoperative surgical plan for the patient that is stored in a database at an appropriate location (e.g., on a server in a wide area network, a local area network, or a global network) where it can be accessed by the surgeon or other care provider, including before and during the actual surgery.

Discussion in this disclosure of actions performed by surgical assistance system 100 may be performed by one or more computing devices (e.g., computing device 102) of surgical assistance system 100, MR visualization device 104, or a combination of the one or more computing devices and MR visualization device 104.

User 109 may use surgical assistance system 100 during a preoperative phase of an orthopedic surgery and an intraoperative phase of the orthopedic surgery. The preoperative phase of the orthopedic surgery may occur before performance of the orthopedic surgery. The orthopedic surgery may be an original orthopedic surgery or an orthopedic revision surgery.

During the preoperative phase of an orthopedic shoulder surgery, data acquisition system 116 of surgical assistance system 100 may obtain patient-specific values of a plurality of physical characteristics of a humeral bone (i.e., a humerus) of a patient. In some examples, a 3-dimensional region at a diaphysis of the humeral bone is partitioned (e.g., by data acquisition system 116) into a first set of blocks and a 3-dimensional region at a metaphysis of the humeral bone is partitioned (e.g., by data acquisition system 116 ) into a second set of blocks. Data acquisition system 116 may use landmarks like greater and lesser tuberosities to define the surgical neck, use deltoid proximal and lateral insertions, and or use the cylindrical shape of the diaphysis to determine locations of the metaphysis and diaphysis. In some examples, data acquisition system 116 may also use statistical shape modeling with anatomic landmarks and pre-labeled regions, and may match the statistical shape model to the patient bone and match the corresponding regions. In some examples, data acquisition system 116 may use average distances described in the medical literature to determine locations of the diaphysis and metaphysis.

The plurality of physical characteristics of the humeral bone may include or consist of (1) an average of Hounsfield units of a first set of blocks that exceed a first threshold, (2) an average of Hounsfield units of the second set of blocks that exceed a second threshold, and (3) an average of Hounsfield units of the first set of blocks that exceed a third threshold different from the first threshold. The first, second, and third thresholds may have various values in different examples. For instance, in one example, the first threshold is equal to 400, the second threshold is equal to 500, and the third threshold is equal to 700. In other examples, the first, second, or third thresholds may have different values, such as 200, 220, 400, 500, 600, 700, 800, 900, 1000, 1100, and so on.

The 3-dimensional region at the diaphysis of the humeral bone may be defined as a part of the humerus between a first fixed distance and a second fixed distance from a proximal tip of the greater tuberosity of the humeral bone. For instance, the first fixed distance may be 8 centimeters and the second fixed distance may be 10 centimeters. In other examples, other fixed distances are possible. Furthermore, in some examples, the distances are not fixed, but instead may be scaled based on overall height of the patient or an overall length of the humerus. In some examples, the 3-dimensional region at the diaphysis of the humeral bone may be determined by locating a most-proximal end of a 2-centimeter-high cylinder of the humeral bone that has parallel outer walls. In some examples, the 3-dimensional region at the metaphysis of the humeral bone may be defined as a part of the humerus between the anatomical neck and a fixed distance from the proximal tip of the greater tuberosity of the humeral bone. For instance, the fixed distance may be 3 or 4 centimeters in average. In some examples, the 3-dimensional region at the metaphysis of the humeral bone is defined as the region between the anatomical neck and the surgical neck of the humeral bone.

In examples where the distances are not fixed, data acquisition system 116 may obtain data generated by performing a dual-energy x-ray absorptiometry (DXA) scan of the patient's humerus. In other examples, data acquisition system 116 may obtain data generated by performing a quantitative computed tomography (QCT) scan of the patient's humerus. Data acquisition system 116 may then analyze the obtained data of the patient's humerus to determine the Hounsfield units of the blocks in the regions of the diaphysis and metaphysis of the humerus.

In other examples, data acquisition system 116 may obtain patient-specific values for other physical characteristics of the humeral bone. For instance, data acquisition system 116 may obtain patient-specific values for one or more Giannotti measurements. A Giannotti measurement is a ratio between a thickness of the cortical bone of the humerus and a total diameter of the humeral diaphysis at a transverse plane passing through the humeral diaphysis.

Different Giannotti measurements correspond to different thresholds used to create different binary masks of the bone. In other words, different Giannotti measurements may be generated when only considering parts of the bone having densities above the corresponding threshold. For example, different Giannotti measurements may be generated using thresholds of 220 Hounsfield Units (HU), 900 HU, 1000 HU, or 1100 HU. After applying the binary mask, data acquisition system 116 may measure the thickness of the cortical and the other parameters. Data acquisition system 116 may obtain the Giannotti measurements based on radiographs, CT scans, or other types of medical imaging data 120.

In another example, data acquisition system 116 may obtain patient-specific values for one or more Tingart measurements. In some examples, a Tingart measurement is one of, or a mean of, a first value, a second value, a third value, and a fourth value. The first value is the lateral cortical thicknesses of the humeral diaphysis at a first level. The second value is the medial cortical thickness of the humeral diaphysis at the first level. The third value is the lateral cortical thickness of the humeral diaphysis at a second level. The fourth value is the medial cortical thicknesses of the humeral diaphysis at the second level. The first level is at a location on the humeral diaphysis where endosteal borders of the lateral and medial cortices of the humerus are first parallel to each other, as determined from the proximal tip of the humerus. The second level is defined as 20 mm distal from the first level. Data acquisition system 116 may obtain the Tingart measurements based on radiographs. Similar to Giannotti measurements, data acquisition system 116 may obtain Tingart measurements using different thresholds used to create different binary masks of the bone.

Furthermore, during the preoperative phase of the orthopedic shoulder surgery, prediction system 118 of surgical assistance system 100 may predict, based on the patient-specific values, a humeral prosthesis for the patient from among a plurality of humeral prostheses. Each humeral prosthesis of the plurality of humeral prostheses may have a different size and/or shape. For example, different humeral prostheses of the plurality of humeral prostheses may have different stem diameters and different stem lengths.

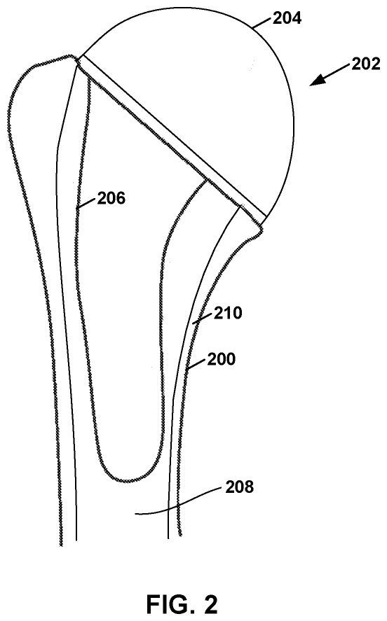

FIG. 2 is a conceptual diagram illustrating an example cross-section of a proximal portion of a humerus 200. As illustrated in the example of FIG. 2, a stemmed humeral prosthesis 202 is implanted into humerus 200. Humeral prosthesis 202 includes a dome-shaped head 204 that may slide within a patient's glenoid cavity or a cavity defined by a glenoid implant. In other examples, humeral prosthesis 202 may include a cup-shaped head, and glenosphere of a glenoid implant may slide within the cup-shaped head of humeral prosthesis 202. Additionally, humeral prosthesis 202 includes a stem 206. Stem 206 is located in an intramedullary canal 208 of humerus 200. Intramedullary canal 208 is located between cortical bone 210 of humerus 200.

Different humeral prostheses may be associated with different index values. For example, a first humeral prosthesis may be associated with an index value of 1, a second humeral prothesis may be associated with an index value of 2, a third humeral prosthesis may be associated with an index value of 3, and so on. In some examples, the index values associated with humeral prostheses increase monotonically with increasing size of the humeral prostheses. To predict a humeral prosthesis for a specific patient, surgical assistance system 100 may determine an index value associated with a humeral implant in the plurality of humeral implants.

Surgical assistance system 100 may determine the index value associated with the humeral implant in one of various ways. For instance, in one example, surgical assistance system 100 may apply an artificial neural network that takes the patient-specific values of the physical characteristics of the patient's humerus as input. The artificial neural network may output an index value associated with the humeral implant.

The artificial neural network may be implemented in one of various ways. For instance, in some examples, the artificial neural network may include an input layer, an output layer, and one or more hidden layers between the input layer and the output layer. In this example, the input layer may include an artificial neuron for each physical characteristic of the plurality of physical characteristics. For example, the input layer may include artificial neurons for (1) an average of Hounsfield units of a first set of blocks that exceed a first threshold, (2) an average of Hounsfield units of the second set of blocks that exceed a second threshold, and (3) an average of Hounsfield units of the first set of blocks that exceed a third threshold different from the first threshold.

In different examples, the output layer of the neural network may include different numbers of artificial neurons. In some examples, the output layer of the neural network includes only a single artificial neuron. The artificial neuron of the output layer outputs an index value of the predicted humeral implant. In other examples, the output layer of the artificial neural network includes a separate artificial neuron corresponding to each humeral prosthesis of the plurality of humeral prostheses. In this example, the artificial neurons of the output layer may output confidence values indicating levels of confidence that the predicted humeral prosthesis should be the humeral prosthesis corresponding to the artificial neuron. In this example, surgical assistance system 100 may select, as the predicted humeral prosthesis, the humeral prosthesis that corresponds to the artificial neuron that generates the highest confidence value.

In this example, the artificial neural network may be trained (e.g., by surgical assistance system 100 or another computing system) based on training data 122. For ease of explanation, this disclosure assumes that surgical assistance system 100 trains the artificial neural network. Training data 122 may include patient training data for a plurality of patients. The training data for a patient may specify patient-specific values for the plurality of physical characteristics. Additionally, training data 122 for the patient may specify an index of humeral prosthesis implanted in the patient. In this example, to train the artificial neural network using a training data set for a patient, prediction system 118 may apply a forward pass through the artificial neural network to generate output data indicating an index of a predicted humeral prosthesis. Prediction system 118 may then compare the index of the predicted humeral prosthesis to the index of the humeral prosthesis indicated by the training data for the patient. Based on this comparison, prediction system 118 may use a backpropagation algorithm to update weight values of artificial neurons in the artificial neural network.

In another example of how prediction system 118 may determine the index value associated with the humeral implant, prediction system 118 may apply an equation to calculate the index value associated with the humeral implant. The equation may be of the following form:

i=a₁x₁+a₂x₂+. . . +aₙxₙ+b (1)

In Equation (1) above, i denotes the index value associated with the humeral implant, a₁, . . . , aₙ are coefficients, x₁, . . . , xₙ are the patient-specific values, and b is a constant.

As noted elsewhere in this disclosure, the physical characteristics of the patient's humerus may include one or more of: (1) an average of Hounsfield units of a first set of blocks that exceed a first threshold, (2) an average of Hounsfield units of the second set of blocks that exceed a second threshold, and (3) an average of Hounsfield units of the first set of blocks that exceed a third threshold different from the first threshold. In such examples, each of these three averages is one of the variables in the equation above. In other words, these three averages may be respective ones of patient-specific values x₁, x₂, . . . , xₙ in Equation (1). In an example where the humeral prostheses are indexed according to an indexing scheme used for humeral prostheses manufactured by Wright Medical Group and the three thresholds are 400, 500, and 700, or other values, such as 200, 220, etc., the coefficients for these three averages are 0.01144, −0.007451, and −0.008808, and the constant is 7.9111.

In some examples, the patient-specific values in Equation (1) may include one or more of an age of the patient, an intramedullary canal diameter of the patient, an average cortical metaphyseal bone density in Hounsfield units that exceed a first threshold (e.g., 200), an average cortical spongious bone density in Hounsfield units that are less than or equal to a second threshold (e.g., 220), a set of shape parameters of a statistical shape model of the humeral bone of the patient, and/or other values. In other words, the patient's age, intramedullary canal diameter, average cortical metaphyseal bone density in Hounsfield units, an average cortical spongious bone density in Hounsfield units, the shape parameters and/or other values may be respective ones of patient-specific values x₁, x₂, . . . xₙ in Equation (1). The intramedullary canal diameter may be referred to as a Tingart M4 parameter because the intramedullary canal diameter because the intramedullary canal diameter is measured at the same location on the humeral bone as the fourth value of the Tingart measurement. In some examples, the patient-specific values in Equation (1) may additionally or alternatively include factors such as gender, diagnosis, and so on.

The shape parameters may be parameters of statistical shape model (SSM) of a humeral bone of the patient. To determine the shape parameters, prediction system 118 may obtain a mean SSM of a humeral bone. The mean SSM of a humeral bone is a model of a humeral bone based on statistics regarding parameters of humeri of multiple people. Additionally, a covariance matrix of data vectors may be determined. Each of the data vectors may comprise coordinates of surface points on humeral bones of individual people (e.g., subpopulation objects). In other words, a vector X=(X₀, X₁, . . . , X₅)^{T} may be determined (e.g., by prediction system 118), where X₀, X₁, . . . , X₅ are coordinates of surface points on the humeral bones of individual people. A covariance matrix Kₓₓ may be determined, where each (i,j) entry of covariance matrix Kₓₓ is a covariance of two of X₀, X₁, . . . , X₅. Next, prediction system 118 may determine eigenvectors and eigenvalues of the covariance matrix. For instance, prediction system 118 may perform a principal component analysis by eigendecomposition of the covariance matrix. Any model of a humeral bone can then be written using the following linear combination of the eigenvectors:

$$x = \overline{x} + {\sum }_{i = 1}^{m} {b}_{i} \sqrt{{λ}_{i}} \times {v}_{i}$$

where x̅ is the statistical mean, bᵢ are shape parameters, λᵢ are the eigenvalues and vᵢ are the eigenvectors.

Prediction system 118 may adapt the mean SSM to the fit the mean SSM to the humeral bone of the actual patient. To fit the mean SSM to the humeral bone of the actual patient, prediction system 118 may perform a minimization procedure. As part of the minimization procedure, prediction system 118 may determine reference points (p_{med-ref}) on a model of the patient's humeral bone (S̨). Additionally, prediction system 118 may compute a first coordinate system of the model of the patient's humeral bone and a second coordinate system of the mean SSM of the humeral bone. Prediction system 118 may then align the first and second coordinate systems. Prediction system 118 may then scale the model of patient's humeral bone to the mean SSM of the humeral bone. Prediction system 118 may then perform a prediction process in which n₀=1 denotes a minimization order and S(b̅) denotes a predicted humeral bone. Shape S may be defined as sets of 3-dimensional coordinates (i.e., S={\tilde{p}ᵢ: \tilde{p}∈R³, i=1, 2, . . . , Nₚ}, where p is the number of points). For i={1, 2, . . . , Nᵢ}, prediction system 118 may repeat the following steps:
1. Perform a rigid registration (T_{rigid}ⁱ) to fit S̨ into Sᵢ₋₁.
2. Minimize a cost function cf at nᵢ≥nᵢ₋₁ and find the corresponding nᵢ shape parameters b̅ᵢ. The cost function cf may be defined as

$$cf ( b ) = OLS = {‖ S - \tilde{S} ( b ) ‖}^{2} = {\sum }_{i = 1}^{{N}_{p}} {‖ p - {\tilde{p}}_{i} ( b ) ‖}^{2}$$

where \tilde{p} is a point on \tilde{S} and p is the closest point to \tilde{p} on S. The notation ∥⋅∥ stands for the L² norm ∥⋅∥₂ (i.e., the Euclidean distance).
3. Generate the new predicted humeral bone Sᵢ. Prediction system 118 may then perform an inverse rigid registration T_{rigid}⁻¹=Πᵢ₌₁^{N}^{ᵢ}(T_{rigid}ⁱ)⁻¹ on the model of the patient's humeral bone. Prediction system 118 may perform an inverse scaling and alignment T_{align}⁻¹×T_{scale}⁻¹ to reposition the model of the patient's humeral bone to an original pose.

As noted above, prediction system 118 may use the shape parameters as patient-specific values in Equation (1). In some examples, the shape parameters may range from ˜−3 to ˜3. In some examples, the shape parameters account for differences between retroversion, inclination, height, radius of curvature, posterior offset, and medial offset of the humeral head between the humeral bone of the patient and the mean humeral bone. In other examples, prediction system 118 does not use all of the shape parameters in Equation (1).

The values of the coefficients may be determined (e.g., by prediction system 118 of surgical assistance system 100 or another computing system) using linear regression. For ease of explanation, this disclosure assumes that prediction system 118 determines the coefficients. In some examples, to determine the values of the coefficients using linear regression, prediction system 118 may use a weighted least square regression technique.

Thus, in some examples, surgical assistance system 100 (e.g., memory 110) may store a plurality of machine-learned coefficients. In such examples, as part of predicting the humeral prosthesis, surgical assistance system 100 may determine a humeral prosthesis index of the predicted humeral implant as a sum of a plurality of elements and a machine-learned constant. Each respective element of the plurality of elements is a multiplication product of a respective machine-learned coefficient in the plurality of machine-learned coefficients and a corresponding physical characteristic in the plurality of physical characteristics of the humeral bone of the patient. The machine-learned coefficients are machine learned using a regression from humeral prosthesis indexes of humeral prostheses implanted in patients with filling ratios of the humeral prostheses that are less than remodeling thresholds for the patients.

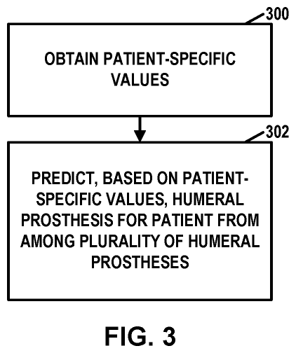

FIG. 3 is a flowchart illustrating an example operation of surgical assistance system 100, in accordance with one or more aspects of this disclosure. In the example of FIG. 3, surgical assistance system 100 (e.g., data acquisition system 116 of surgical assistance system 100) obtains patient-specific values (300). The patient specific values may include patient-specific values of a plurality of physical characteristics of a humeral bone of a patient. For instance, in some examples, the plurality of physical characteristics includes (i) an average cortical metaphyseal bone density in Hounsfield units that exceed a first threshold, (ii) an average cortical spongious bone density in Hounsfield units that are less than or equal to a second threshold, and (iii) one or more shape parameters based on changes to shape parameters of a mean statistical shape model (SSM) of a generic humeral bone to conform the mean SSM to the humeral bone of the patient. In some the patient-specific values further include one or more of an age of the patient, a gender of the patient, or a diagnosis of the patient. Surgical assistance system 100 may obtain the patient-specific values in accordance with any of the examples provided elsewhere in this disclosure.

Additionally, in the example of FIG. 3, surgical assistance system 100 predicts, based on the patient-specific values, a humeral prosthesis for the patient from among a plurality of humeral prostheses (302). In some examples, the predicted humeral prosthesis has a filling ratio less than a remodeling threshold for the patient. As noted elsewhere in this disclosure, the filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient. The remodeling threshold for the patient is a filling ratio above which the humeral prosthesis would cause bone remodeling in the patient. As an example of predicting the humeral prosthesis, surgical assistance system 100 determines a humeral prosthesis index of the predicted humeral implant as a sum of a plurality of elements and a machine-learned constant. In this example, each respective element of the plurality of elements being a multiplication product of a respective machine-learned coefficient in the plurality of machine-learned coefficients and a corresponding physical characteristic in the plurality of physical characteristics of the humeral bone of the patient. In some examples, surgical assistance system 100 may verify that the filling ratio is less than the remodeling threshold after determining the humeral prosthesis index.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1. A method comprising:
obtaining, by a surgical assistance system, patient-specific values of a plurality of physical characteristics of a humeral bone of a patient; and
predicting, by the surgical assistance system, based on the patient-specific values, a humeral prosthesis for the patient from among a plurality of humeral prostheses, wherein the predicted humeral prosthesis has a filling ratio less than a remodeling threshold for the patient, wherein:
the filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient, and
the remodeling threshold for the patient is a filling ratio above which the predicted humeral prosthesis would cause bone remodeling in the patient.

2. The method of claim 1, wherein:
the method comprises storing a plurality of machine-learned coefficients, and
predicting the humeral prosthesis comprises determining a humeral prosthesis index of the predicted humeral implant as a sum of a plurality of elements and a machine-learned constant, each respective element of the plurality of elements being a multiplication product of a respective machine-learned coefficient in the plurality of machine-learned coefficients and a corresponding physical characteristic in the plurality of physical characteristics of the humeral bone of the patient.

3. The method of claim 2, wherein the machine-learned coefficients are machine learned using a regression from humeral prosthesis indexes of humeral prostheses implanted in patients with filling ratios of the humeral prostheses that are less than remodeling thresholds for the patients.

4. The method of claim 1, wherein:
a region at a diaphysis of the humeral bone is partitioned into a first set of blocks, a region at a metaphysis of the humeral bone is partitioned into a second set of blocks, obtaining the patient-specific values for the plurality of physical characteristics comprises:
calculating a first value as an average of Hounsfield units of the first set of blocks that exceed a first threshold,
calculating a second value as an average of Hounsfield units of the second set of blocks that exceed a second threshold, and
calculating a third value as an average of Hounsfield units of the first set of blocks that exceed a third threshold different from the first threshold.

5. The method of claim 4, wherein the plurality of physical characteristics consists of the first value, the second value, and the third value.

6. The method of claim 1, wherein predicting the humeral prosthesis comprises predicting, by the surgical assistance system, the humeral prosthesis based on the patient-specific values of the plurality of physical characteristics of the humeral bone and based on an age of the patient.

7. The method of claim 1, wherein the patient-specific values regarding the physical characteristics of the humeral bone of the patient include one or more shape parameters based on changes to shape parameters of a mean statistical shape model (SSM) of a generic humeral bone to conform the mean SSM to the humeral bone of the patient

8. A method comprising:
obtaining, by a surgical assistance system, patient-specific values of a plurality of patient-specific values, wherein:
the plurality of patient-specific values includes patient-specific values of a plurality of physical characteristics of a humeral bone of a patient, and
the plurality of physical characteristics includes (i) an average cortical metaphyseal bone density in Hounsfield units that exceed a first threshold, (ii) an average cortical spongious bone density in Hounsfield units that are less than or equal to a second threshold, and (iii) one or more shape parameters of a statistical shape model (SSM) of the humeral bone of the patient; and
predicting, by the surgical assistance system, based on the patient-specific values, a humeral prosthesis for the patient from among a plurality of humeral prostheses.

9. The method of claim 8, wherein the patient-specific values further include one or more of an age of the patient, a gender of the patient, or a diagnosis of the patient.

10. The method of claim 8, wherein:
the method comprises storing a plurality of machine-learned coefficients, and
predicting the humeral prosthesis comprises determining a humeral prosthesis index of the predicted humeral implant as a sum of a plurality of elements and a machine-learned constant, each respective element of the plurality of elements being a multiplication product of a respective machine-learned coefficient in the plurality of machine-learned coefficients and a corresponding physical characteristic in the plurality of physical characteristics of the humeral bone of the patient.

11. A computing system comprising:
a memory configured to store medical imaging data; and
processing circuitry configured to:
obtain patient-specific values of a plurality of physical characteristics of a humeral bone of a patient and
predict, based on the patient-specific values, a humeral prosthesis for the patient from among a plurality of humeral prostheses, wherein the predicted humeral prosthesis has a filling ratio less than a remodeling threshold for the patient, wherein:
the filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient, and
the remodeling threshold for the patient is a filling ratio above which the predicted humeral prosthesis would cause bone remodeling in the patient.

12. (canceled)

13. A non-transitory computer-readable data storage medium having instructions stored thereon that, when executed, cause a computing system to:
obtain patient-specific values of a plurality of physical characteristics of a humeral bone of a patient and
predict, based on the patient-specific values, a humeral prosthesis for the patient from among a plurality of humeral prostheses, wherein the predicted humeral prosthesis has a filling ratio less than a remodeling threshold for the patient, wherein:
the filling ratio of the humeral prosthesis is a ratio of: (i) a radial distance from a lengthwise central axis of a stem of the humeral prosthesis to an outer surface of the stem, to (ii) a radial distance from the lengthwise central axis of the stem to an inner surface of an intramedullary canal of a humerus of the patient, and
the remodeling threshold for the patient is a filling ratio above which the predicted humeral prosthesis would cause bone remodeling in the patient.

14. The computing system of claim 11,
wherein the memory stores a plurality of machine-learned coefficients, and
the processing circuitry is configured to, as part of predicting the humeral prosthesis, determine a humeral prosthesis index of the predicted humeral implant as a sum of a plurality of elements and a machine-learned constant, each respective element of the plurality of elements being a multiplication product of a respective machine-learned coefficient in the plurality of machine-learned coefficients and a corresponding physical characteristic in the plurality of physical characteristics of the humeral bone of the patient.

15. The computing system of claim 14, wherein the machine-learned coefficients are machine learned using a regression from humeral prosthesis indexes of humeral prostheses implanted in patients with filling ratios of the humeral prostheses that are less than remodeling thresholds for the patients.

16. The computing system of claim 11, wherein:
a region at a diaphysis of the humeral bone is partitioned into a first set of blocks,
a region at a metaphysis of the humeral bone is partitioned into a second set of blocks,
the processing circuitry is configured to, as part of obtaining the patient-specific values for the plurality of physical characteristics:
calculate a first value as an average of Hounsfield units of the first set of blocks that exceed a first threshold,
calculate a second value as an average of Hounsfield units of the second set of blocks that exceed a second threshold, and
calculate a third value as an average of Hounsfield units of the first set of blocks that exceed a third threshold different from the first threshold.

17. The computing system of claim 16, wherein the plurality of physical characteristics consists of the first value, the second value, and the third value.

18. The computing system of claim 11, wherein predicting the humeral prosthesis comprises predicting, by the surgical assistance system, the humeral prosthesis based on the patient-specific values of the plurality of physical characteristics of the humeral bone and based on an age of the patient.

19. The computing system of claim 11, wherein the patient-specific values regarding the physical characteristics of the humeral bone of the patient include one or more shape parameters based on changes to shape parameters of a mean statistical shape model (SSM) of a generic humeral bone to conform the mean SSM to the humeral bone of the patient.

20. The non-transitory computer-readable data storage medium of claim 13, wherein the instructions that cause the computing system to predict the humeral prosthesis comprises instructions that, when executed, cause the computing system to determine a humeral prosthesis index of the predicted humeral implant as a sum of a plurality of elements and a machine-learned constant, each respective element of the plurality of elements being a multiplication product of a respective machine-learned coefficient in a plurality of machine-learned coefficients and a corresponding physical characteristic in the plurality of physical characteristics of the humeral bone of the patient.

21. The non-transitory computer-readable data storage medium of claim 20, wherein the machine-learned coefficients are machine learned using a regression from humeral prosthesis indexes of humeral prostheses implanted in patients with filling ratios of the humeral prostheses that are less than remodeling thresholds for the patients.
